(19) United States
(12) Patent Application Publication
(10) Pub. No.: US 2020/0276129 A1
(43) Pub. Date: Sep. 3, 2020

(54) TABLET CONTAINING VALSARTAN AND SACUBITRIL

(71) Applicant: ALFRED E. TIEFENBACHER (GMBH & CO. KG), Hamburg (DE)

(72) Inventors: Bala Ramesha Chary RALLABANDI, Hyderabad (Telangana) (IN); Vamshi Ramana PRATHAP, Mallial (IN); Krishna Mohan GOLLAPUDI, Peddapurum Mandal (IN); Bala Subramanian VELUSAMY, Hyderabad (IN); Srimannarayana BANDLA, Hyderabad (IN); Hendrik SCHLEHAHN, Hamburg (DE); Dieter RUCHATZ, Hamburg (DE)

(73) Assignee: ALFRED E. TIEFENBACHER (GMBH & CO. KG), Hamburg (DE)

(21) Appl. No.: 16/646,904

(22) PCT Filed: Oct. 12, 2018
(86) PCT No.: PCT/EP2018/077953

§ 371 Date: Mar. 12, 2020

Related U.S. Application Data
(60) Provisional application No. 62/591,270, filed on Nov. 28, 2017.

(30) Foreign Application Priority Data
Oct. 13, 2017 (IN) 201711036502

(51) Int. Cl. A61K 9/20; A61K 9/28; A61K 31/41; A61K 31/216

(52) U.S. Cl. CPC A61K 9/2095; A61K 31/216; A61K 31/41; A61K 9/28

(57) ABSTRACT

The present invention relates to a tablet for oral administration containing valsartan and sacubitril, preferably as sodium salts or as a complex of valsartan disodium and sacubitril monosodium, preferably LCZ696. The tablet is prepared by dry-granulation or direct compression and contains a mesoporous inorganic stabilizer, e.g. mesoporous silica (Syloid®).

The present invention relates to a process for preparing a valsartan and sacubitril-containing tablet as well as to a valsartan and sacubitril-containing tablet that is prepared by dry-granulation or direct compression.

The combination valsartan and sacubitril is marketed under the tradename Entresto® in the form of film-coated tablets for the prevention of heart failure in patients with chronic heart failure. Entresto® contains the drug combination in the form of a cocrystal consisting of valsartan disodium, sacubitril monosodium and 2.5 molecules water. The cocrystal has been designated as LCZ696; its preparation and physical/chemical properties are described in WO 2007/056546 and in Tetrahedron Letters 2012, 53, 275-276. In the Tetrahedron Letters, it is further reported that a desolvated crystalline form exists because the crystalline structure of LCZ696 is maintained up to the melting temperature (around 138° C.), in spite of the fact that two water molecules are lost during the heating.

Various polymorphic forms, pseudopolymorphic forms of LCZ696, i.e. crystalline forms in which the cocrystal contains either more molecules or less molecules of water than 2.5 molecules, and amorphous forms of LCZ696 are known, which are described in WO 2016/037552, WO 2016/049663, WO 2016/051393, WO 2016/125123, WO 2016/151525, WO 2016/201238, WO 2017/009784 and WO 2017/012917.

The Entresto® film-coated tablet is an immediate-release tablet that contains, besides LCZ696, microcrystalline cellulose, low-substituted hydroxypropyl cellulose, crospovidone, magnesium stearate, talc and colloidal silicon dioxide as pharmaceutical excipients. Three strengths of the tablet are marketed, which contain, on the basis of the free acid weight of the drugs, 24 mg/26 mg, 49 mg/51 mg and 97 mg/103 mg of sacubitril/valsartan. WO 2009/061713 discloses an immediate-release tablet containing LCZ696 prepared by direct compression or dry-granulation. In the preparation of the tablet, moisture, excessive heat and high shear forces should be avoided in order to prevent amorphization as well as dissociation of the drug components of LCZ696. According to the EMA Entresto® Assessment report 2015, the commercially available tablet is prepared by a dry-granulation roller compaction process because it was a more robust manufacturing method than the direct compression method that was employed for the manufacture of the tablets used in the early clinical studies.

WO 2017/000864 describes a direct compression method for preparing a tablet containing LCZ696, in which a mixture of the drug, a hydrophilic diluent, a binder and a disintegrant is subjected to compression.

WO 2017/012600 discloses a tablet containing a physical mixture of sacubitril or a pharmaceutically acceptable salt thereof and valsartan or a pharmaceutically acceptable salt thereof that can be prepared by direct compression, dry-granulation or wet-granulation. The tablets are very sensitive to moisture, so that packaging under nitrogen atmosphere is recommended in order to prevent the degradation of the drugs.

WO 2017/037596 discloses an amorphous solid dispersion of LCZ696 prepared by rotational distillation, spray-drying or freeze-drying a solution containing LCZ696 and a pharmaceutical excipient such as a polymer or silica (e.g. Syloid®) or magnesium aluminometasilicate (e.g. Neusilin®). In addition, the preparation of amorphous LCZ696 is described, wherein crystalline LCZ696 is dissolved in an appropriate organic solvent that is subsequently removed by evaporation.

U.S. Pat. No. 5,217,996 discloses a process for the preparation of sacubitril and its pharmaceutically acceptable salts, in particular, the preparation of the monosodium salt of sacubitril. WO 02/06253 describes various salts of valsartan, inter alia, the disodium salt in crystalline or amorphous form. Amorphous and crystalline forms of valsartan are described in WO 2004/083192.

LCZ696, valsartan disodium and sacubitril monosodium cannot be easily processed due to their poor flowability. Moreover, the amorphous forms of valsartan disodium and LCZ696 as well as sacubitril and its pharmaceutically acceptable salts, such as sacubitril monosodium, are very hygroscopic solids. These substances become deliquescent and sticky when exposed to air humidity.

It is well known that the handling and the formulation of hygroscopic and deliquescent, sticky active ingredients into solid pharmaceutical formulations is difficult and requires extensive precautions. When water absorption occurs during manufacturing, the consequences include processing problems such as stickiness, clumping, poor release from punches, poor flow characteristics and poor compressibility. Moreover, the physico-mechanical properties and appearance of solid dosage forms comprising a hygroscopic or deliquescent active ingredient are often insufficient, especially after storage. For example, an insufficient tablet hardness as well as unacceptable crumbling and cracking or even liquifying of the solid dosage forms may occur.

The objective underlying the present invention was the provision of a process for preparing a sacubitril and valsartan-containing tablet in which the processability of the active ingredients is improved. It was a further objective to provide an optionally film-coated tablet in which valsartan and sacubitril are chemically and physically (no amorphization, recrystallization or (pseudo)polymorph conversion) stable. These objectives are attained by the subject matter as defined in the claims.

The tablet of the present invention is an immediate-release tablet for oral administration, preferably a film-coated tablet. The tablet of the present invention contains:
a) valsartan or a pharmaceutically acceptable salt thereof and sacubitril or a pharmaceutically acceptable salt thereof as active ingredients,
b) a mesoporous inorganic stabilizer, and
c) a pharmaceutically acceptable excipient,
   wherein the process comprises the method steps:
i) mixing the active ingredients with the mesoporous inorganic stabilizer and with the pharmaceutically acceptable excipient, and
ii) subjecting the blend obtained in step (i) to compression to obtain the tablet,
   or
iii) mixing the active ingredients with the mesoporous inorganic stabilizer and with the pharmaceutically acceptable excipient,
iv) subjecting the blend obtained in step (iii) to compaction,
v) milling the compacted blend obtained in step (iv) to obtain granules,
vi) optionally mixing the granules obtained in step (v) with the pharmaceutically acceptable excipient and optionally with the mesoporous inorganic stabilizer, and
vii) subjecting the granules obtained in step (v) or the blend obtained in step (vi) to compression to obtain the tablet,
   wherein method steps (i) to (vii) are performed in an environment of a relative humidity of not more than 50%, preferably not more than 45%, and most preferably not more than 40%.

It was found that the stability of the active ingredients is improved if the process is performed in an environment of a relative humidity of not more than 50%, preferably not more than 45% and most preferably not more than 40%.

In a preferred embodiment of the present invention, the tablet is prepared by a process comprising the method steps:
i) mixing the active ingredients and the mesoporous inorganic stabilizer,
ii) mixing the blend obtained in step (i) with the pharmaceutically acceptable excipient and optionally with the mesoporous inorganic stabilizer, and
iii) subjecting the blend obtained in step (ii) to compression to obtain the tablet,
   or
iv) mixing the active ingredients and the mesoporous inorganic stabilizer,
v) mixing the blend obtained in step (iv) with the pharmaceutically acceptable excipient,
vi) subjecting the blend obtained in step (v) to compaction,
vii) milling the compacted blend obtained in step (vi) to obtain granules,
viii) optionally mixing the granules obtained in step (vii) with the pharmaceutically acceptable excipient and optionally with the mesoporous inorganic stabilizer, and
ix) subjecting the granules obtained in step (vii) or the blend obtained in step (viii) to compression to obtain the tablet,
   wherein method steps (i) to (ix) are performed in an environment of a relative humidity of not more than 50%, preferably not more than 45%, and most preferably not more than 40%.

It was found that the flowability and stability of the active ingredients may be further improved by preparing a preblend consisting of the active ingredients and the mesoporous inorganic stabilizer (method step (i) or (iv) of the preferred process of the present invention). The mesoporous inorganic stabilizer serves as a dehydrating agent and a glidant. A mesoporous material is a material containing pores with diameters of 2-50 nm.

Suitable mesoporous silica products are commercially available under the tradename Syloid®. Syloid® is a hydrated silica because it contains more hydroxy groups at the surface compared to fumed (colloidal) silica. Other mesoporous silica products are commercially available under the tradename Aeroperl® 300 Pharma, which consists of bead-like granules of colloidal silica, and Parteck® SLC. As an alternative to mesoporous silica, mesoporous magnesium aluminometasilicate may be used, e.g. the magnesium aluminometasilicates marketed under the tradename Neusilin®. A further alternative is mesoporous magnesium carbonate, which is available under the tradename Upsalite®.

Typically, the weight ratio of the active ingredients to the mesoporous inorganic stabilizer in method step (i) or (iv) of the preferred embodiment is 1:1 to 50:1, preferably 5:1 to 20:1, and most preferably 8:1 to 15:1. It was found that, compared to the 1:1-weight ratio used in the examples of WO 2017/037596 for preparing the amorphous solid dispersion of LCZ696, the amount of the mesoporous inorganic stabilizer required in method step (i) or (iv) of the preferred embodiment can be reduced, if the process is performed in an environment of a relative humidity of not more than 50%, preferably not more than 45% and most preferably not more than 40%; the lower the relative humidity of the environment, the lower the amount of mesoporous inorganic stabilizer required for protecting the active ingredients from moisture.

It is preferred that in method step (ii) or (viii) of the preferred embodiment of the process of the present invention, the blend obtained in step (i) or the granules obtained in step (vii) are mixed with the pharmaceutically acceptable excipient and with the mesoporous inorganic stabilizer.

According to a preferred embodiment of the present invention, the tablet comprises the active ingredients in a ratio (mol/mol) of 1:1, wherein the active ingredients are preferably valsartan disodium and sacubitril monosodium. Alternatively, the active ingredients are in the form of a complex of valsartan disodium and sacubitril monosodium.

Valsartan disodium, sacubitril monosodium or the complex of valsartan disodium and sacubitril monosodium may be in amorphous form. Preferably, the complex is LCZ696. Alternatively, valsartan disodium, sacubitril monosodium or the complex of valsartan disodium and sacubitril monosodium may be in a crystalline form, preferably the complex is LCZ696 or a polymorphic form or pseudopolymorphic form thereof. The expression “pseudopolymorphic form” relates to crystalline hydrates of the complex of valsartan disodium and sacubitril monosodium other than the hemipentahydrate LCZ696, which contain either more water molecules or less water molecules than 2.5 molecules in the crystal lattice.

The pharmaceutical excipient contained in the tablet of the present invention may be selected from diluents, disintegrants, lubricants and glidants.

Examples of diluents include microcrystalline cellulose, calcium hydrogen phosphate, lactose (anhydrous or monohydrate), mannitol, calcium carbonate, carboxymethylcellulose calcium, starch, pregelatinized starch, magnesium carbonate, silicified microcrystalline cellulose, powdered cellulose, sorbitol, xylitol and magnesium aluminometasilicate, whereby microcrystalline cellulose and mannitol are preferably contained. Examples of disintegrants include croscarmellose sodium, sodium starch glycolate, polyvinylpolypyrrolidone (crospovidone) and low-substituted hydroxypropyl cellulose (L-HPC), whereby crospovidone and L-HPC are preferred. As glidants fumed (colloidal) silicon dioxide, talc, magnesium silicate and the like may be used, while magnesium stearate, calcium stearate, stearic acid, sodium stearyl fumarate and glycerol dibehenate are examples of suitable lubricants. Multifunctional excipients may also be included, e.g. coprocessed microcrystalline cellulose (diluent)/hydroxypropyl methylcellulose (binder)/crospovidone (disintergrant) (e.g. PanExcea® MHC300G) or coprocessed tricalcium phosphate (diluent)/polyvinylpyrrolidone (binder) (e.g. Innophos® TCP-DC).

The process of the present invention is either a direct compression process (method steps (i)-(ii) or, in the preferred embodiment, method steps (i)-(iii)) or a dry-granulation process (method steps (iii)-(vii) or, in the preferred embodiment, method steps (iv)-(ix)), meaning that the active ingredients, the mesoporous inorganic stabilizer and the pharmaceutically acceptable excipients are processed in solid form and that the blends obtained in the process of the present invention are powdery blends. The process of the present invention is performed without the use of water or organic solvents.

The compaction in method step (iv) (method step (vi) in the preferred embodiment) is preferably a slugging process. If roller compaction is used, it is preferred that the roller compaction is performed twice in order to provide sufficiently hard granules. Moreover, it is preferred to perform both the slugging process and the roller compaction twice in order to decrease the proportion of fine material, thereby improving the flowability of the obtained granules. In addition, it is preferred that the active ingredients are used in non-micronized form in the process of the present invention because particle size reduction increases the surface and the fines portion, and, thus, the hygroscopicity of the active ingredients. Preferably, the particle size distribution of the active substances is adjusted to (as determined by the wet method described in the experimental part):

direct compression method:
sacubitril monosodium: Dᵥ50=40-100 μm, Dᵥ90=100-300 μm
valsartan disodium: Dᵥ50=20-100 μm, Dᵥ90=50-300 μm
LCZ696: Dᵥ50=20-50 μm, Dᵥ90=50-300 μm,
dry-granulation method:
sacubitril monosodium: Dᵥ50=20-100 μm, Dᵥ90=100-300 μm
valsartan disodium: Dᵥ50=20-100 μm, Dᵥ90=50-300 μm
LCZ696: Dᵥ50=20-50 μm, Dᵥ90=50-300 μm.

The tablet of the present invention is preferably coated with a moisture-barrier film-coating in order to increase the hygroscopic stability of the tablet; for example, the tablets may be coated with an aqueous dispersion of Opadry. It was found that the stability of the active ingredients is further improved, if the tablet is pre-warmed at a temperature of 40° C. to 80° C., preferably 50° C. to 70° C., for a sufficient time (usually at least 0.25 hour, preferably 0.5 to 2 hours) before coating and if the coated tablet is heated at a temperature of 40° C. to 80° C., preferably 50° C. to 70° C., for a sufficient time (usually at least 0.25 hour, preferably 0.5 to 4 hours) until the water content of the film-coated tablet is 5% or below (loss on drying).

The tablets of the present invention are contained in blister-patches or bottles made, for example, from PVC, PVDC, PCTFE, COC, PET, PA, Alu, PE or PP and combinations or multilayer films thereof. These packages may comprise a moisture barrier layer and/or they may be packed together with desiccants. The desiccant may be optionally integrated into a layer of a packaging, for example blister film, sachet or bottle.

The following examples are intended to further illustrate the present invention.

### EXAMPLES

In the examples, amorphous LCZ696 having a particle size distribution of Dᵥ50=5-100 μm and Dᵥ90=20-500 μm, crystalline sacubitril monosodium having a particle size distribution of Dᵥ50=10-150 μm and Dᵥ90=50-500 μm and amorphous valsartan disodium having a particle size distribution of Dᵥ50=10-150 μm and Dᵥ90=50-500 μm were used. The particle size was determined by the following wet method using Mastersizer 2000:

### 1.1 Dispersion Medium Preparation

1.1.1 Prepare a saturated solution of the API in n-heptane (saturated n-heptane solution).
1.1.2 Prepare a mixture of silicone oil and the saturated n-heptane solution in the ratio of (60:40) v/v. This solution is used as dispersion medium.

### 1.2 Dispersant Preparation

1.2.1 Prepare a solution by dissolving 0.5 ml of span-85 in 500.0 ml n-heptane.

### 1.3 Procedure

1.3.1 Sample preparation: transfer about 50 mg of sample into a dry 20.0 ml stoppered Nessler cylinder, add 1.0 ml of dispersant and gently mix with glass rod.
1.3.2 To the above solution add 3.0 ml of saturated n-heptane solution followed by 6.0 ml of silicone oil, the solution is mixed well and sonicated for 2 min.
1.3.3 The measurement cell is filled with isopropyl alcohol when the instrument is not used. Before starting the analysis, rinse the cell twice with isopropyl alcohol followed by n-heptane.
1.3.4 Fill the measurement cell with dispersion medium, add all the sample solution from the Nessler cylinder into sample tank. Allow the sample to circulate for about 30 to 60 seconds monitoring the obscuration rate.
1.3.5 Wait for obscuration rate is constant and start the measurement.
1.3.6 Measure the particle size of the sample preparation three times.
1.3.7 Perform the measurement for 3 individual preparations. Report as average results (Instrument average) of 3 individual preparations.
1.3.8 System suitability: RSD for Dᵥ90 for three individual preparations should not be more than 15%.

### XRD Method

The XRD measurements were performed using X-ray source with Cu K-alpha radiation, Empyrean system (or equivalent), PIXcel detector, divergence slit 0.25° fixed, anti-scattering slit 0.5°, soller slits 2×0.02 radians, Nickel filter to suppress back ground and Cu K-beta components, current 40 mA, voltage 45 kV, 2°-40°2θ, spinning 30 RPM, step size 0.013° with total measurement time 1 Hr at room temperature.

### Sample Preparation for XRD

1. Grind about two tablets to fine powder using Agate mortar and pestle gently. Prepare the sample (approx. 350 mg) using PANalytical sample preparation kit by ‘Back loading technique’. The sample surface should be smooth and in parallel to sample holder surface. Clean the outer edges of the holder with tissue paper to avoid sample contaminations,
2. Place the prepared sample holder carefully on the sample stage of the XRD instrument and analyze as per the above XRD method conditions at room temperature.

### Determination of the Water Content

The water content (loss on drying) of the film-coated tablet was determined as described in chapter 2.5.12 or 2.2.32 of the European Pharmacopeia 9.0.

### Examples 1-5 (Dry-Granulation)

| | Ex. 1 | Ex. 2 | Ex. 3 | Ex. 4 | Ex. 5 |
|---|---|---|---|---|---|
| | [mg] | [mg] | [mg] | [mg] | [mg] |
| Pre-mixing | | | | | |
| LCZ696 | — | — | 224.728* | 224.728* | — |
| Sacubitril sodium | 103.937* | 103.937* | — | — | 103.937* |
| Valsartan disodium | 119.015* | 119.015* | — | — | 119.015* |
| Hydrated silica | 21.000 | 21.000 | 21.000 | 21.000 | 21.000 |
| (Syloid AL1-FP) | | | | | |
| Blending and Slugging | | | | | |
| Microcrystalline cellulose | 23.253 | 31.253 | 21.477 | 48.477 | 34.048 |
| (Comprecel 102) | | | | | |
| Low substituted | 30.000 | 30.000 | 30.000 | 21.000 | 30.000 |
| hydroxypropyl cellulose | | | | | |
| (L-HPC LH11) | | | | | |
| Crospovidone Type A | 20.000 | 15.000 | 20.000 | 10.000 | 14.00 |
| (Polyplasdone XL) | | | | | |
| Magnesium stearate | 4.000 | 4.000 | 4.000 | 4.000 | 4.000 |
| Lubrication | | | | | |
| Mannitol | 44.795 | 44.795 | 44.795 | 44.795 | 44.000 |
| (Pearlitol 200 SD) | | | | | |
| Crospovidone Type A | 18.000 | 15.000 | 18.000 | 10.000 | 14.00 |
| (Polyplasdone XL) | | | | | |
| Hydrated silica | 4.000 | 4.000 | 4.000 | 4.000 | 4.000 |
| (Syloid AL1-FP) | | | | | |
| Talc | 4.000 | 4.000 | 4.000 | 4.000 | 4.000 |
| Magnesium stearate | 8.000 | 8.000 | 8.000 | 8.000 | 8.000 |
| Core Tablet | 400.000 | 400.000 | 400.000 | 400.000 | 400.000 |
| Film-Coating | | | | | |
| Opadry 00F540020 Pink | 16.000 | 16.000 | 16.000 | 16.000 | 16.000 |
| Water | q.s. | q.s. | q.s. | q.s. | q.s. |
| Film-Coated Tablet | 416.000 | 416.000 | 416.000 | 416.000 | 416.000 |

*Contains 97 mg sacubitril and 103 mg valsartan

### Manufacturing Process:

### Step-1: Premixing

Sacubitril sodium and Valsartan disodium ((or) LCZ 696) along with Silica (Syloid) were cosifted through suitable screen and blended.

### Step-2: Blending and Slugging

To the step-1 premix powder mixture, previously sifted Microcrystalline cellulose (Comprecel® M102D+), Low substituted hydroxypropyl cellulose (L-HPC LH11), crospovidone type A (Polyplasdone® XL) and Magnesium stearate were added and blended. This powder mixture was compressed into slugs with suitable hardness. The slugs were sized using suitable screen. If necessary, the obtained granules were again compressed into slugs, which were subsequently sized until the fine percentage (ASTM #60 passings) reached below 40. The second slugging cycle improved the flowability of the granules due the reduction of the fine material proportion.

### Step-3: Lubrication

To the step-2 granules, previously sifted Mannitol (Pearlitol® 200SD), crospovidone type A (Polyplasdone® XL), Magnesium stearate, Silica (Syloid®) and Talc were added and blended.

### Step-4: Tableting

The step-3 blend was compressed into tablets in a rotary tableting machine.

### Step-5: Film-Coating

Step-4 core tablets were coated with Opadry aqueous dispersion to get approximately 4% weight gain.

### Example 6 (Direct Compression)

| | Example. 6 |
|---|---|
| | [mg] |
| Pre Mixing | |
| Sacubitril Sodium | 102.939* |
| Valsartan disodium | 118.266* |
| Hydrated silica (Syloid 244FP) | 8.000 |
| Blending and Lubrication | |
| Microcrystalline cellulose (Avicel PH 200) | 96.795 |
| Low substituted hydroxypropyl cellulose (L-HPC LH11) | 20.000 |
| Crospovidone (Polyplasdone XL) | 30.000 |
| Hydrated colloidal silica (Syloid 244FP) | 8.000 |
| Talc | 4.000 |
| Magnesium stearate | 12.000 |
| Core Tablet | 400.000 |
| Film-Coating | |
| Opadry 00F540020 Pink | 16.000 |
| Water | q.s. |
| Film-Coated Tablet | 416.000 |

*Contains 97 mg sacubitril and 103 mg valsartan

### Manufacturing Process:

### Step-1: Premixing

Sacubitril sodium and Valsartan disodium ((or) LCZ 696) along with Silica (Syloid) were cosifted through suitable screen and blended.

### Step-2: Blending and Lubrication

To the step-1 premix powder mixture, previously sifted Microcrystalline cellulose (Avicel®PH 200), Low substituted hydroxypropyl cellulose (L-HPC LH11), crospovidone type A (Polyplasdone® XL), Silica (Syloid®), Talc and Magnesium stearate were added and blended.

### Step-3: Tableting

The step-2 blend was compressed into tablets in a rotary tableting machine.

### Step-4: Film-Coating

Step-3 core tablets were coated with Opadry aqueous dispersion to get approximately 4% weight gain.

### Examples 7-9 (Dry-Granulation)

| | Ex. 7 | Ex. 8 | Ex. 9 |
|---|---|---|---|
| | [mg] | [mg] | [mg] |
| A. Pre-mixing | | | |
| Sacubitril sodium | 103.937* | 51.969⁺ | 25.984^{#} |
| Valsartan disodium | 119.015* | 59.508⁺ | 29.754^{#} |
| Hydrated silica (Syloid AL1-FP) | 21.000 | 10.500 | 5.250 |
| B. Blending and Slugging | | | |
| Microcrystalline cellulose | 31.253 | 15.627 | 7.813 |
| (Comprecel 102) | | | |
| Low substituted hydroxypropyl | 30.000 | 15.000 | 7.500 |
| cellulose (L-HPC LH11) | | | |
| Crospovidone Type A | 30.000 | 15.000 | 7.500 |
| (Polyplasdone XL) | | | |
| Mannitol (Pearlitol 200 SD) | 44.795 | 22.398 | 11.199 |
| Hydrated silica (Syloid AL1-FP) | 4.000 | 2.000 | 1.000 |
| Magnesium stearate | 4.000 | 2.000 | 1.000 |
| C. Lubrication | | | |
| Talc | 4.000 | 2.000 | 1.000 |
| Magnesium stearate | 8.000 | 4.000 | 2.000 |
| Core Tablet | 400.000 | 200.000 | 100.000 |
| D. Film-Coating | | | |
| Opadry 00F540020 Pink | 16.000 | — | — |
| Opadry 00F520031 Yellow | — | 8.000 | — |
| Opadry 00F500003 Purple | — | — | 4.000 |
| Water | q.s. | q.s. | q.s. |
| Film-Coated Tablet | 416.000 | 208.000 | 104.000 |

*Contains 97 mg sacubitril and 103 mg valsartan
⁺Contains 49 mg sacubitril and 51 mg valsartan
^{#}Contains 24 mg sacubitril and 26 mg valsartan

### Manufacturing Process:

### Step-1: Premixing

Stage A ingredients were cosifted using suitable screens in suitable equipment. This cosifted blend was mixed in a suitable blender.

### Step-2: Blending and Slugging

To the step 1 premix powder, stage B ingredients, which were sifted using suitable screen, were added and mixed. This blend was compacted into slugs using rotary tableting machine with suitable hardness.

### Step-3: Milling

The step-2 compacts were milled through Quadro Comill using suitable screen.

### Step-4: Blending

To the step-3 milled granules, previously sifted stage C ingredients were added and mixed in a suitable blender.

### Step-5: Compression

The step-4 blend was compressed into tablets in a rotary tableting machine using suitable punches with suitable hardness.

### Step-6: Film-Coating

Step-5 core tablets were coated with Opadry aqueous dispersion to get approximately 4% weight gain.

The tablets showed good physical and chemical stability. No cracking of the tablets could be observed after storage in an Alu-Alu blister for three months at 40° C./75% relative humidity (RH). In addition, no crystallization of the amorphous LCZ696 and the amorphous valsartan disodium, and no polymorph conversion or amorphization of the crystalline sacubitril monosodium could be detected by powder XRD.

### Examples 10-12 (Dry-Granulation)

| | Ex. 10 | Ex. 11 | Ex. 12 |
|---|---|---|---|
| | [mg] | [mg] | [mg] |
| Pre-mixing | | | |
| Sacubitril sodium | 25.598^{#} | 51.196⁺ | 102.392* |
| Valsartan disodium | 28.294^{#} | 56.589⁺ | 113.177* |
| Hydrated silica (Syloid AL1-FP) | 5.250 | 10.500 | 21.000 |
| Blending and Compaction | | | |
| Microcrystalline cellulose | 9.608 | 19.215 | 38.431 |
| Low substituted hydroxypropyl | 7.500 | 15.000 | 30.000 |
| cellulose | | | |
| Crospovidone | 7.500 | 15.000 | 30.000 |
| Mannitol | 11.250 | 22.500 | 45.000 |
| Magnesium stearate | 1.000 | 2.000 | 4.000 |
| Lubrication | | | |
| Hydrated silica (Syloid AL1-FP) | 1.000 | 2.000 | 4.000 |
| Magnesium stearate | 2.000 | 4.000 | 8.000 |
| Talc | 1.000 | 2.000 | 4.000 |
| Core Tablet weight | 100.000 | 200.000 | 400.000 |
| Film-Coating | | | |
| Opadry 00F540020 Pink | — | — | 16.000 |
| Opadry 06F520005 Yellow | — | 10.000 | — |
| Opadry 06F500001 Purple | 5.000 | — | — |
| Water | q.s. | q.s. | q.s. |
| Film-Coated Tablet weight | 105.000 | 210.000 | 416.000 |

*Contains 97.2 mg sacubitril and 102.8 mg valsartan
⁺Contains 48.6 mg sacubitril and 51.4 mg valsartan
^{#}Contains 24.3 mg sacubitril and 25.7 mg valsartan

### Manufacturing Process:

### Step-1: Sifting

Sacubitril Sodium, Valsartan disodium and Silica were cosifted through a suitable screen. Sift microcrystalline cellulose, Low-substituted hydroxypropyl cellulose, Crospovidone, Mannitol and Magnesium stearate through a suitable screen.

### Step-2: Pre-Mixing

Transfer Sacubitril Sodium, Valsartan disodium and Silica from step 1 into blender and mix for sufficient duration at suitable blender speed.

### Step-3: Blending

Mix sifted microcrystalline cellulose, Low-substituted hydroxypropyl cellulose, Crospovidone, Mannitol and Magnesium stearate from step 1 with premixed material from step 2 in suitable blender for sufficient duration at suitable blender speed.

### Step-4: Compaction

Compact the material from step 3 using roll compactor or using compression machine with suitable process parameters and machine settings.

### Step-5: Milling

Mill the compacts from step 4 using suitable mill with suitable speed.

### Step-6: Sifting of Extra-Granular Materials

Sift Silicon dioxide, Magnesium stearate and Talc through a suitable screen.

### Step-7: Lubrication

Load the milled granules from step 5 and sifted materials from step 6 into suitable blender and mix for sufficient duration at suitable blender speed.

### Step-8 Compression

Compress the tablets of 24/26 mg, 49/51 mg and 97/103 mg using suitable tooling and in-process parameters.

### Step-9: Coating

Prepare Coating dispersion by dispersing Opadry® purple for 24/26 mg, Opadry® yellow for 49/51 mg and Opadry® pink for 97/103 mg in Purified water. Stir for 45 minutes and load tablets from step 8 into coating machine. Pre-warm the tablets at bed temperature of 55±10° C. for sufficient duration. Coat the tablets by using suitable coating machine process parameters. Coat the tablets of each strength by using suitable coating machine process parameters. Warm the coated tablets at bed temperature of 55±10° C. for sufficient duration.

### Stability of the Film-Coated Tablet Described in Example 12 Having Different Water Contents (Adjusted by the Drying Time of the Coated Tablets):

| | Initial | | | |
|---|---|---|---|---|
| Drying | water | Storage | | |
| time | content | conditions | Packaging | XRD |
| No | 6.58% | 50° C./75% | Clear PVC/ | complex formed |
| drying | | RH one month | Aclar-Alu | water content: 6.78% |
| | | | Alu-Alu | complex formed |
| | | | | water content: 6.26% |
| 30 min | 5.46% | | Clear PVC/ | complex formed |
| | | | Aclar | water content: 6.04% |
| | | | Alu-Alu | complex formed |
| | | | | water content: 5.73% |
| 60 min | 5.10% | | Clear PVC/ | complex formed |
| | | | Aclar | water content: 5.83% |
| | | | Alu-Alu | complex formed |
| | | | | water content: 5.28% |
| 120 min | 4.97% | | Clear PVC/ | complex formed |
| | | | Aclar | water content: 5.75% |
| | | | Alu-Alu | no API polymorphic |
| | | | | form conversion |
| | | | | water content: 4.78% |
| 240 min | 4.18% | | Clear PVC/ | no API polymorphic |
| | | | Aclar | form conversion |
| | | | | Water content: 4.36% |
| | | | Alu-Alu | no API polymorphic |
| | | | | form conversion. |
| | | | | water content 4.01% |
| 120 min | 4.97% | 50° C./75% | Alu-Alu | no API polymorphic |
| | | RH two months | | form conversion. |
| | | | | (Sacubitril/ |
| | | | | Valsartan complex |
| | | | | content is below |
| | | | | detection limit) |
| | | | | water content: 4.51 |
| 240 min | 4.18% | | Alu-Alu | no API polymorphic |
| | | | | form conversion. |
| | | | | (Sacubitril/ |
| | | | | Valsartan complex |
| | | | | content is below |
| | | | | detection limit) |
| | | | | water content: 4.08 |

1. A process for preparing an optionally film-coated tablet containing:
a) valsartan or a pharmaceutically acceptable salt thereof and sacubitril or a pharmaceutically acceptable salt thereof as active ingredients,
b) a mesoporous inorganic stabilizer, and
c) a pharmaceutically acceptable excipient,
wherein the process comprises the method steps:
i) mixing the active ingredients with the mesoporous inorganic stabilizer and with the pharmaceutically acceptable excipient, and
ii) subjecting the blend obtained in step (i) to compression to obtain the tablet,
or
iii) mixing the active ingredients with the mesoporous inorganic stabilizer and with the pharmaceutically acceptable excipient,
iv) subjecting the blend obtained in step (iii) to compaction,
v) milling the compacted blend obtained in step (iv) to obtain granules,
vi) optionally mixing the granules obtained in step (v) with the pharmaceutically acceptable excipient and optionally with the mesoporous inorganic stabilizer, and
vii) subjecting the granules obtained in step (v) or the blend obtained in step (vi) to compression to obtain the tablet,
wherein method steps (i) to (vii) are performed in an environment of a relative humidity of not more than 50%, preferably not more than 45%, and most preferably not more than 40%.

2. The process according to claim 1, wherein the process comprises the method steps:
i) mixing the active ingredients and the mesoporous inorganic stabilizer,
ii) mixing the blend obtained in step (i) with the pharmaceutically acceptable excipient and optionally with the mesoporous inorganic stabilizer, and
iii) subjecting the blend obtained in step (ii) to compression to obtain the tablet,
or
iv) mixing the active ingredients and the mesoporous inorganic stabilizer,
v) mixing the blend obtained in step (iv) with the pharmaceutically acceptable excipient,
vi) subjecting the blend obtained in step (v) to compaction,
vii) milling the compacted blend obtained in step (vi) to obtain granules,
viii) optionally mixing the granules obtained in step (vii) with the pharmaceutically acceptable excipient and optionally with the mesoporous inorganic stabilizer, and
ix) subjecting the granules obtained in step (vii) or the blend obtained in step (viii) to compression to obtain the tablet,
wherein method steps (i) to (ix) are performed in an environment of a relative humidity of not more than 50%, preferably not more than 45%, and most preferably not more than 40%.

3. The process according to claim 2, wherein the weight ratio of the active ingredients to the mesoporous inorganic stabilizer in method step (i) or (iv) is 1:1 to 50:1, preferably 5:1 to 20:1, and most preferably 8:1 to 15:1.

4. The process according to claim 2, wherein in step (ii) or (viii), the blend obtained in step (i) or the granules obtained in step (vii) are mixed with the pharmaceutically acceptable excipient and with the mesoporous inorganic stabilizer.

5. The process according to claim 1, wherein the tablet comprises the active ingredients in a ratio (mol/mol) of 1:1.

6. The process according to claim 1, wherein the active ingredients are valsartan disodium and sacubitril monosodium, or wherein the active ingredients are in the form of a complex of valsartan disodium and sacubitril monosodium.

7. The process according to claim 6, wherein the valsartan disodium or the complex of valsartan disodium and sacubitril monosodium is in an amorphous form.

8. The process according to claim 7, wherein the complex of valsartan disodium and sacubitril monosodium is LCZ696.

9. The process according to claim 6, wherein the valsartan disodium is in a crystalline form.

10. The process according to claim 6, wherein the complex of valsartan disodium and sacubitril monosodium is in a crystalline form.

11. The process according to claim 10, wherein the complex of valsartan disodium and sacubitril monosodium is LCZ696 or a polymorphic form or pseudopolymorphic form thereof.

12. The process according to claim 1, wherein the mesoporous inorganic stabilizer is selected from silica, magnesium aluminometasilicate and magnesium carbonate.

13. A tablet prepared by the process according to claim 1.

14. The tablet according to claim 13, wherein the pharmaceutical excipient is selected from diluents, disintegrants, lubricants and glidants.

15. The tablet according to claim 13, wherein the tablet is coated with a moisture-barrier film coating.

16. The tablet according to claim 13, wherein the optionally film-coated tablet has a water content (loss on drying) of 5% or below.

17. The process according to claim 3, wherein in step (ii) or (viii), the blend obtained in step (i) or the granules obtained in step (vii) are mixed with the pharmaceutically acceptable excipient and with the mesoporous inorganic stabilizer.

18. The process according to claim 2, wherein the tablet comprises the active ingredients in a ratio (mol/mol) of 1:1.

19. The process according to claim 3, wherein the tablet comprises the active ingredients in a ratio (mol/mol) of 1:1.

20. The process according to claim 4, wherein the tablet comprises the active ingredients in a ratio (mol/mol) of 1:1.
